(19) United States
(12) Patent Application Publication
(10) Pub. No.: US 2009/0247653 A1
(43) Pub. Date: Oct. 1, 2009

(54) Configurations And Methods of SNG Production

(75) Inventors: Ravi Ravikumar, Lancaster, CA (US); Giorgio Sabbadini, Costa Mesa, CA (US)

Correspondence Address: FISH & ASSOCIATES, PC;ROBERT D. FISH, 2603 Main Street, Suite 1000, Irvine, CA, 92614-6232, US

(73) Assignee: FLUOR TECHNOLOGIES CORPORATION, Aliso Viejo, CA (US)

(21) Appl. No.: 12/295,715

(22) PCT Filed: Apr. 5, 2007
(86) PCT No.: PCT/US07/08575

§ 371 Date: Jun. 11, 2009

Related U.S. Application Data
(60) Provisional application No. 60/790,241, filed on Apr. 6, 2006.

(51) Int. Cl. C07C 27/00; B01J 7/00

(52) U.S. Cl. 518/708; 422/188; 422/198

(57) ABSTRACT

SNG plants according to the inventive subject matter include one or more methanation reactors that produce a primary methanation product that is cooled to a temperature sufficient to condense water, which is removed in a separator. So produced dried methanation product is then split to provide a reflux stream to the methanation reactors and a feed stream to an adiabatic trim reactor. Most preferably, the plant comprises at least two methanation reactors that are operated in series, wherein the first reactor receives the recycle stream and wherein the second reactor receives a portion of the first methanation reactor effluent and a portion of the first methanation reactor feed.

This application claims priority to our copending provisional patent application with the Ser. No. 60/790241, which was filed Apr. 6, 2006, and which is incorporated by reference herein.

### FIELD OF THE INVENTION

The field of the invention is production of substitute natural gas (SNG) from various carbonaceous materials via gasification.

### BACKGROUND OF THE INVENTION

With rapidly rising prices for natural gas, production of SNG from coal or petcoke has become increasingly economically attractive. Most commonly, SNG is produced from such materials using gasification followed by a water gas shift conversion to produce a syngas that has a H2/CO ratio of about 3. The following reactions I and II summarize the methanation process using CO, CO2, and H2:

CO+3H2=CH4+H2O (I)

CO2+4H2=CH4+2H2O (II)

As the above reactions are highly exothermic, multiple reaction stages are frequently required to control the temperature within limits tolerable for the nickel catalyst. A typical plant 100 for SNG production is depicted in Prior Art FIG. 1, in which lignite is gasified using a moving bed gasification process (not shown) at a production volume of about 170 MMSCFD SNG. In such plants, a methanation unit receives sulfur free syngas from sour shift/Rectisol units (not shown) with a H2/CO ratio of about 3. The reaction system typically includes two primary reactors 110 and 120 in series, and a downstream isothermal trim reactor 130. Here, the first primary reactor 110 receives about half of the fresh preheated feed 102 as stream 102A and further receives a compressed gas recycle stream 104E′ from the second primary reactor 120 to achieve a desirable inlet temperature and an acceptable outlet temperature. The effluent 104A from the first primary reactor 110 is cooled in cooler 160 and blended with the preheated balance 102B of the fresh feed 102 to form stream 104B that is then routed to the second primary reactor 120. The second primary reactor effluent 104C is cooled in steam generator 162 to produce steam and stream 104D. A first portion of the cooled effluent 104D is recycled as stream 104E to the first primary reactor 110 via recycle compressor 170, while a second portion 104F is further cooled in cooler 164 to form stream 104G that is then fed to the isothermal trim reactor 130. The synthetic natural gas exiting trim reactor 130 is then cooled in cooler 140 and dried in drier 150 to form the final SNG product.

There are numerous catalysts known in the art to support such methanation reaction, and such catalysts are commonly commercially available (e.g., Johnson Matthey, Sud-Chemie, Haldor Topsoe, etc.). Further known systems for generation of SNG are described, for example, in U.S. Pat. No. 4,235,044. As the final SNG product often has a relatively high heating value per SCF, SNG is typically blended with natural gas in the pipeline to conform with pipeline and combustion standards. While such configurations and processes often provide a relatively reliable manner of SNG production, no significant efforts were made to substantially improve the economics of such process. In further known systems, as described in U.S. Pat. No. 4,133,825, the synthesis gas is conditioned and CO2 removed to thus allow for use of an adiabatic methanation, and in yet other known systems, steam recycling is employed to reduce carbon formation as described in U.S. Pat. No. 4,005,996.

Therefore, while numerous methods of SNG production are known in the art, all or most of them suffer from one or more disadvantages. Among other things, heretofore known configurations and methods often require relatively large volumes of catalyst and expensive process equipment. Consequently, there is still a need to provide improved configurations and methods of SNG production.

### SUMMARY OF THE INVENTION

The inventors have discovered that SNG production plants and processes can be run more efficiently and at reduced capital costs by removing water from the trim reactor feed and recycle stream, which in turn allows for increased trim reactor inlet temperature and reduced catalyst volume. Thus, such water removal advantageously increases overall yield of CH4 and further allows replacement of the isothermal trim reactor with a significantly less expensive adiabatic trim reactor.

In one aspect of the inventive subject matter, a plant for production of synthetic natural gas comprises a syngas source that provides a feed gas having a H2 to CO ratio of between 2.5 to 3.5. A first and a second primary reactor are fluidly coupled to the syngas source such that a first portion of the feed gas is delivered to the first primary reactor and a second portion of the feed gas is delivered to the second primary reactor, wherein the first primary reactor is further fluidly coupled to the second primary reactor such that a combination of the second portion of the feed gas and an effluent of the first primary reactor is fed into the second primary reactor. Contemplated plants further comprise a cooler that receives and cools the effluent of the second primary reactor to a temperature sufficient to condense water in the effluent of the second primary reactor, and a separator that is fluidly coupled to the second primary reactor and that separates the water from the effluent of the second primary reactor to thereby produce an at least partially dried effluent. Most preferably, a recycle conduit is coupled to the separator and the first primary reactor such that a first portion of the dried effluent is fed to the first primary reactor, and an adiabatic trim reactor is fluidly coupled to the separator and receives a second portion of the dried effluent.

In such plants, it is especially preferred that an acid gas removal unit removes acid gas (e.g., H2S) and contaminants (e.g., COS, HCN, NH3, organic thiols, metal carbonyls) from the feed gas, and that an upstream shift unit increases H2 content in the feed gas. Further contemplated plants will preferably include a heater that heats the feed gas to temperature of between 400° F. and 900° F. and a second heater that heats the dried effluent.

Viewed from a different perspective, a plant for production of synthetic natural gas includes a condensation system (typically including a steam generator, a cooler, and a separator) that cools the reactor effluent from an upstream methanation reactor to thereby form water condensate and an at least partially dried reactor effluent. In such plants, a first conduit will then deliver a first portion of the at least partially dried effluent to the upstream methanation reactor, and a second conduit delivers a second portion of the at least partially dried effluent to an adiabatic trim reactor to thus produce the synthetic natural gas from the at least partially dried effluent. In such plants, it is generally preferred that a heater heats the dried reactor effluent prior to entry into the adiabatic trim reactor.

Typically, the syngas in such plants will be produced by gasification of a carbonaceous feed and have a H2 to CO ratio of between 2.5 to 3.5. It is still further preferred that such plants include a second upstream methanation reactor that receives effluent from the upstream methanation reactor, and that further receives a portion of the feed gas. Most typically, a compressor compresses the first portion of the at least partially dried effluent to the operating pressure of the upstream methanation reactor.

Therefore, in another aspect of the inventive subject matter, a method of producing synthetic natural gas will include a step of converting in a methanation reactor syngas having a H2 to CO ratio of between 2.5 to 3.5 to a primary methanation product. In another step, the primary methanation product is cooled to a temperature effective to condense water, which is separated from the primary methanation product to thereby form an at least partially dried methanation product and water. In a still further step, a first portion of the at least partially dried methanation product is fed to the methanation reactor and a second portion of the at least partially dried methanation product is fed to a (most preferably adiabatic) trim reactor.

Most preferably, the second portion of the at least partially dried methanation product is heated before feeding the second portion to the trim reactor, and/or the step of converting the syngas is performed in at least two methanation reactors that are fluidly coupled to each other in series, wherein the at least two methanation reactors receive a portion of the syngas (ratio between first and second portion is typically between 1:1 and 1:10).

Various objects, features, aspects and advantages of the present invention will become more apparent from the following detailed description of preferred embodiments of the invention and the accompanying drawing.

### BRIEF DESCRIPTION OF THE DRAWING

Prior Art FIG. 1 depicts an exemplary known configuration for a plant for SNG production.

FIG. 2 depicts an exemplary configuration for a plant for SNG production according to the inventive subject matter.

### DETAILED DESCRIPTION

The inventors have surprisingly discovered that SNG production plants and processes can be significantly improved by removing at least a portion of water from the trim reactor feed and recycle stream to the primary methanation reactor to thereby increase conversion of CO and CO2 to CH4. Moreover, it should be recognized that the configurations and methods according to the inventive subject matter also allow use of less expensive process equipment, and particularly allow replacement of the isothermal trim reactor with an adiabatic trim reactor. Still further, it is pointed out that removal of a portion of the water also allows the trim reactor inlet temperature to be increased and to maintain SNG product: quality while at the same time to reduce catalyst volume in the reactor.

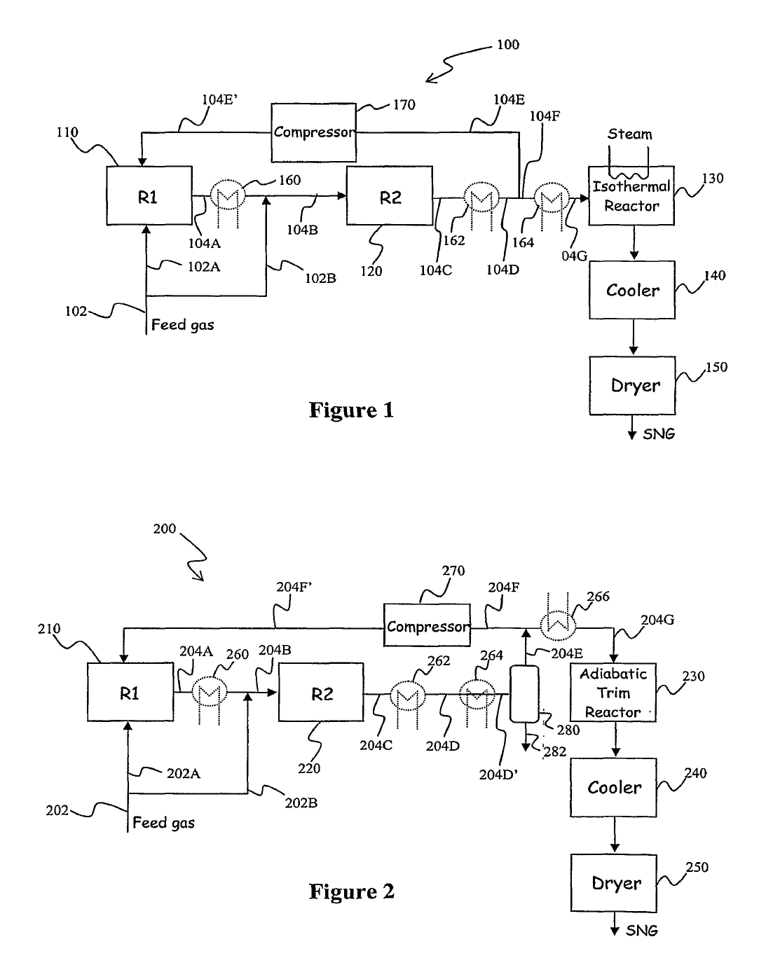

One exemplary configuration according to the inventive subject matter is depicted in FIG. 2. Here, plant 200 includes first and second primary methanation reactors 210 and 220 and (preferably an adiabatic) trim reactor 230. In such plant, a first portion 202A of the feed gas 202 is heated (heater not shown) and fed to the first primary reactor 210, while a second portion 202B of the feed gas 202 is combined with cooled first primary reactor effluent to serve as feed 204B for the second primary reactor (the first primary reactor 210 produces hot effluent gas 204A, which is cooled in steam generator 260 to form the cooled first primary reactor effluent). The second primary reactor 220 produces hot effluent gas 204C, which is cooled in steam generator 262 to form cooled stream 204D. Cooled stream 204D is further reduced in temperature in cooler 264 to allow for water condensation in stream 204D′. A separator 280 separates the condensate 282 from the at least partially dried effluent 204E, which is then split into two streams, recycle stream 204F and trim reactor feed 204G.

Recycle stream 204F is increased in pressure to operating pressure of the first primary methanation reactor by compressor 270 to form compressed stream 204F′, while the trim reactor feed 204G is first heated in heater 266 (e.g., heat exchanger using heat from the effluent gases) to a temperature suitable for operation of the adiabatic trim reactor 230. The reactor effluent of reactor 230 is then processed as in Prior Art FIG. 1 by cooling in cooler 240 and dryer 250 to produce the final SNG product. It should be especially noted that the reduction of the water content in the reactor feed significantly increases the conversion of CO and CO2 to CH4, thus reducing the CO, H2 and CO2 in the trim reactor effluent, and thereby increases the heating value of SNG.

Therefore, it should be particularly appreciated that contemplated plants for production of synthetic natural gas include a condensation system that cools the reactor effluent from an upstream methanation reactor train to thereby form (predominantly water) condensate and an at least partially dried reactor effluent, wherein a first portion of the at least partially dried effluent is recycled to the upstream methanation reactor (most preferably the first methanation reactor), and wherein a second portion of the at least partially dried effluent is routed (typically after heating) to an adiabatic trim reactor that is configured to produce the synthetic natural gas from the at least partially dried effluent. Thus, methods of producing SNG are particularly contemplated in which in a methanation reactor syngas (having a H2 to CO ratio of between 2.5 to 3.5) is converted to a primary methanation product. The so produced primary methanation product is then cooled to a temperature effective to condense water, which is separated from the primary methanation product to thereby form an at least partially dried methanation product. In such methods, it is typically preferred that a first portion of the at least partially dried methanation product is recycled to the methanation reactor and that a second portion of the at least partially dried methanation product is fed to a (most typically adiabatic) trim reactor.

Contemplated configurations and processes are particularly advantageous where SNG is produced from coal and/or petcoke, and when prices for natural gas are above $7-8 per MMBTU (current projections expect price fluctuations between about 9-12$ per MMBTU between March 2006 and December 2008 as estimated in the natural gas market update by the Federal Energy Regulatory Commission). In another example, SNG production from coal is economically attractive in the Illinois area as this area has vast high sulfur coal reserves. It is estimated that a typical coal to SNG plant will produce about 110 MMSCFD SNG from about 6300 tpd (dry) coal. Furthermore, SNG is storable in pipelines under pressure and therefore allows operation of SNG plants on a base load mode, which avoids the need for cycling (turning down during off-peak periods) as compared to most electric power plants. Contemplated plants and configurations are also ecologically advantageous as emissions from a coal to SNG plant are minimal compared to an IGCC (Integrated Gasification Combined Cycle) plant. Still further, it should be noted that net carbon dioxide emission is minimal as such plants can produce CO2 as byproduct suitable for sequestration or for enhanced oil recovery. Similarly, as the feed gas to the SNG plant is already desulfurized, overall sulfur capture is expected to be in excess of 99.99%.

With respect to the feed gas it is generally contemplated that feed gases having a H2 to CO ratio of about 2.5 to about 3.5, and most preferably of about 3 are deemed suitable. The term “about” where used herein in conjunction with a numeral refers to a +/−10% range of that numeral. Thus, gasification of most carbonaceous and/or organic feed is considered suitable for use herein, and most preferably coal and/or petcoke is used as starting material for gasification. Typically, such gasification is performed using well known configurations and methods. It is further particularly preferred that the gases from the gasification will be treated prior to entering the SNG plant. For example, the gas from the gasification reactor may be subjected to a shift conversion to convert a portion of the CO to H₂. Also, in most cases acid gases will be removed from the gas using selective or non-selective methods well known in the art. Preferably, acid gases are removed using a (preferably physical) solvent based process. For example, cold methanol may be employed to remove the undesired . components, including H2S, COS, organic thiols, HCN, NH3, metal carbonyls, etc. The loaded solvent can then be regenerated by flashing and stripping (and optionally heating) using conventional processes.

Suitable primary methanation reactors include all currently known reactors, which can be operated using catalysts well known in the art. For example, especially suitable catalysts include low-temperature catalysts comprising an alumina matrix with oxides of nickel and rare earth metals. Therefore, continuous operation temperature will generally be limited to a temperature of less than 900° F.

Furthermore, heating and cooling of the various process streams can be achieved in numerous manners, and all currently known manners are deemed suitable for use herein. Most preferably, cooling the streams will recover at least some of the energy of the exothermic reactions, and all known cooling processes with energy recovery are deemed suitable for use herein. However, especially preferred cooling processes will provide steam (e.g., to drive steam turbines or to provide heating to solvent regeneration processes) or heat for heat exchange with a heater. Cooling of the methanation reactor effluent to condense water is preferably performed in two stages, wherein the first stage produces steam and wherein the second stage may provide heating to a waste heat circuit. Regardless of the manner of cooling, it is contemplated that the temperature of the cooled methanation reactor effluent is between about 60° F. and 200° F., more typically between 70° F. and 170° F., and most typically between 80° F. and 140° F. Depending on the particular water content and cooling temperature, it is contemplated that at least 20%, more typically 40%, even more typically at least 60%, and most typically at least 80% of the water in the methanation reactor effluent is removed.

Furthermore, it is generally preferred that the feed gas is split about equally between the first and second primary reactors. However, where appropriate, the ratio may also be other than 50-50, and feed ratios between about 20-80 to about 80-20 are also contemplated suitable for use herein. Similarly, the recycle streams to the first primary reactor from the separator and/or the second primary reactor may vary considerably. However, it is typically preferred that the ratio between the recycle stream and the feed stream to the trim reactor is between 1:1 and 1:10. Cooling and dehydration of the SNG may be performed in numerous manners and all known manners are deemed suitable for use herein. For example, cooling may be performed using heat exchangers that may or may not be thermally coupled to one or more components of the plant. Dehydration may be performed using various known processes, and especially preferred dehydration processes are glycol-based or employ molecular sieves.

Thus, specific embodiments and applications of configurations and methods of SNG production have been disclosed. It should be apparent, however, to those skilled in the art that many more modifications besides those already described are possible without departing from the inventive concepts herein. The inventive subject matter, therefore, is not to be restricted except in the spirit of the present disclosure. Moreover, in interpreting the specification and contemplated claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Furthermore, where a definition or use of a term in a reference, which is incorporated by reference herein is inconsistent or contrary to the definition of that term provided herein, the definition of that term provided herein applies and the definition of that term in the reference does not apply.

1. A plant for production of synthetic natural gas, the plant comprising:
a syngas source configured to provide a feed gas having a H2 to CO ratio of between 2.5 to 3.5;
a first and a second primary reactor fluidly coupled to the syngas source such that a first portion of the feed gas is delivered to the first primary reactor and a second portion of the feed gas is delivered to the second primary reactor;
wherein the first primary reactor is further fluidly coupled to the second primary reactor such that a combination of the second portion of the feed gas and an effluent of the first primary reactor is fed into the second primary reactor;
a cooler configured to receive and cool an effluent of the second primary reactor to a temperature sufficient to condense water in the effluent of the second primary reactor;
a separator that is fluidly coupled to the second primary reactor and that is configured to separate the water from the effluent of the second primary reactor to thereby produce an at least partially dried effluent;
a recycle conduit coupled to the separator and the first primary reactor such that a first portion of the at least partially dried effluent is fed to the first primary reactor; and
an adiabatic trim reactor fluidly coupled to the separator and configured to receive a second portion of the at least partially dried effluent.

2. The plant of claim 1 further comprising an acid gas removal unit configured to remove acid gas and contaminants from the feed gas.

3. The plant of claim 2 wherein the acid gas removal unit is configured to operate with a physical solvent, wherein the acid gas is H2S, and wherein the contaminant comprises at least one of COS, HCN, NH3, an organic thiol, and a metal carbonyl.

4. The plant of claim 1 further comprising a shift unit upstream of the first and second primary reactor and configured to increase H2 content in the feed gas.

5. The plant of claim 1 further comprising a heater that is configured to heat the feed gas to temperature of between 400° F. and 900° F.

6. The plant of claim 1 further comprising a second heater that is configured to heat the at least partially dried effluent.

7. The plant of claim 1 wherein the first and second primary reactors further comprise a low-temperature catalyst.

8. A plant for production of synthetic natural gas, the plant comprising:
a condensation system that cools reactor effluent from an upstream methanation reactor to thereby form water condensate and an at least partially dried reactor effluent;
a first conduit that is configured to deliver a first portion of the at least partially dried effluent to the upstream methanation reactor; and
a second conduit that is configured to deliver a second portion of the at least partially dried effluent to an adiabatic trim reactor that is configured to produce the synthetic natural gas from the at least partially dried effluent.

9. The plant of claim 8 further comprising a syngas source configured to provide a feed gas having a H2 to CO ratio of between 2.5 to 3.5.

10. The plant of claim 8 further comprising a second upstream methanation reactor configured to receive effluent from the upstream methanation reactor.

11. The plant of claim 10 wherein the second upstream methanation reactor is further configured to receive a portion of the feed gas.

12. The plant of claim 8 further comprising a compressor that compresses the first portion of the at least partially dried effluent to operating pressure of the upstream methanation reactor.

13. The plant of claim 8 wherein the condensation system comprises a steam generator, a cooler, and a separator.

14. The plant of claim 8 further comprising a heater that is configured to heat the at least partially dried reactor effluent.

15. A method of producing synthetic natural gas, the method comprising:
converting in a methanation reactor syngas having a H2 to CO ratio of between 2.5 to 3.5 to a primary methanation product;
cooling the primary methanation product to a temperature effective to condense water and separating the water from the primary methanation product to thereby form an at least partially dried methanation product and water; and
feeding a first portion of the at least partially dried methanation product to the methanation reactor and feeding a second portion of the at least partially dried methanation product to a trim reactor.

16. The method of claim 15 wherein the trim reactor is an adiabatic trim reactor.

17. The method of claim 15 further comprising a step of heating the second portion of the at least partially dried methanation product before feeding the second portion to the trim reactor.

18. The method of claim 15 wherein the step of converting the syngas is performed in at least two methanation reactors that are fluidly coupled to each other in series, and wherein the at least two methanation reactors receive a portion of the syngas.

19. The method of claim 15 wherein the first portion and the second portion of the at least partially dried methanation product have a ratio of between 1:1 and 1:10.

20. The method of claim 15 further comprising a step of removing acid gas from the syngas.
